(19) United States
(12) Patent Application Publication
(10) Pub. No.: US 2022/0257888 A1
(43) Pub. Date: Aug. 18, 2022

(54) RESPIRATORY THERAPY DEVICE AND COUPLING DEVICE FOR COUPLING AT LEAST TWO RESPIRATORY THERAPY DEVICES

(71) Applicant: Loewenstein Medical Technology S.A., Luxembourg (LU)

(72) Inventors: Jan SCHATTNER, Pinneberg (DE); Christof GOEBEL, Hamburg (DE)

(21) Appl. No.: 17/595,710

(22) PCT Filed: May 18, 2020
(86) PCT No.: PCT/EP2020/025229

§ 371 Date: Nov. 23, 2021

(30) Foreign Application Priority Data
May 25, 2019 (DE) 10 2019 003 710.3

(51) Int. Cl. A61M 16/00

(52) U.S. Cl. CPC A61M 16/0057; A61M 2205/8206

(57) ABSTRACT

The present invention relates to a respiratory therapy device (1) for producing a breathable air stream for carrying out respiratory therapy, comprising a housing system (2) for housing device components. The housing system (2) comprises two mounting surfaces (12, 22) so that the respiratory therapy device (1) can be mounted and operated as intended in two mounting positions (3, 4). The invention also relates to a coupling system (200) for coupling at least two respiratory therapy devices (1), as well as to an apparatus comprising said coupling system (200), and to a coupling system (200) for receiving a respiratory therapy device (1).

The present invention relates to a respiratory therapy device for generating a respiratory air stream for carrying out respiratory therapy. The respiratory therapy device comprises at least one housing system for accommodating device components. The invention moreover relates to a coupling system for coupling at least two such respiratory therapy devices.

To achieve the most convenient use of respiratory therapy devices for ventilation or cough assistance, the space requirements and the accessibility and operability of the device are very important. Particularly when ventilating a patient in bed or in a wheelchair, there is often only very little space available in which to place the device. At the same time, however, the device also has to be placed in such a way that the operating elements are easily accessible and the information shown on a display can be clearly seen. An additional consideration is that the position of the user changes, and the position of the device should likewise be able to adapt.

In light of the above, it is the object of the present invention to improve the use of respiratory therapy devices and, in particular, to make their use and operation more convenient even when there is limited space available. In particular, the use of two respiratory therapy devices, for example for ventilation and for cough assistance, should also be made more convenient.

This object is achieved with the respiratory therapy device of claim 1 and with the coupling system according to claim 28. Developments and advantageous embodiments are the subject matter of the dependent claims. Further advantages and features will become clear from the general description and from the description of the exemplary embodiments.

The respiratory therapy device according to the invention serves to generate a respiratory air stream for carrying out respiratory therapy. The respiratory therapy device comprises at least one housing system for accommodating device components. The housing system comprises at least two mounting surfaces. The respiratory therapy device can therefore be mounted and preferably operated as intended in at least two mounting positions. In particular, the respiratory therapy device is operated as intended in the at least two mounting positions.

The respiratory therapy device according to the invention affords many advantages. A considerable advantage is afforded by the housing system with the mounting surfaces, such that the respiratory therapy device can be mounted and also operated in different mounting positions. Thus, depending on the available space and on the surrounding circumstances, the optimal mounting position can be sought. For example, for a person who is lying down, a different mounting position than the one for a person who is standing can be adopted easily and quickly.

The housing system preferably comprises at least one first mounting surface for at least one first mounting position. The housing system preferably comprises at least one second mounting surface for at least one second mounting position. In particular, at least one mounting surface is provided for each mounting position.

The first mounting surface is preferably suitable and designed for a horizontal mounting position. The second mounting surface is preferably suitable and designed for a vertical mounting position. In the horizontal mounting position, the respiratory therapy device lies in particular on the first mounting surface. In the vertical mounting position, the respiratory therapy device stands in particular on the second mounting surface.

Such embodiments permit a particularly simple and convenient change between a horizontal and a vertical use of the respiratory therapy device. Depending on the accessibility requirements, and depending on the location of use and the position of the user, the device can be used either vertically or horizontally. To change between the mounting positions, the device simply has to be placed a different way and, for example, tilted from the vertical to the horizontal position, or vice versa. The user can thus easily adapt the device to his needs by means of adjusting the mounting position by setting the device upright or tilting it.

In the horizontal mounting position, a mounting height of the respiratory therapy device is in particular less than a length and/or width of the first mounting surface. In the vertical mounting position, a mounting height of the respiratory therapy device is in particular greater than a length and/or width of the second mounting surface. The accessibility and the space requirement of the device can thus be particularly advantageously adapted through the choice of the mounting position.

In the horizontal mounting position, the mounting height corresponds in particular to the length and/or width of the second mounting surface. In the vertical mounting position, the mounting height corresponds in particular to the length and/or width of the first mounting surface. In particular, the first mounting surface is longer and/or wider than the second mounting surface. In particular, the first mounting surface and the second mounting surface are designed to be of equal width. It is also possible that the first mounting surface and the second mounting surface are designed to be of equal length. In particular, the mounting surfaces correspond to the base area of the respiratory therapy device in the mounting position on the respective mounting surface.

The horizontal mounting position is defined in particular in that the maximum and/or mean height of the respiratory therapy device, in particular of the housing system, is less than the maximum and/or mean length and/or maximum and/or mean width of the respiratory therapy device, in particular of the housing system. In the horizontal mounting position, the maximum and/or mean height of the respiratory therapy device, in particular of the housing system, is in particular less than the maximum and/or mean length and/or maximum and/or mean width of the first mounting surface on which the respiratory therapy device is set in the horizontal mounting position.

The vertical mounting position is defined in particular in that the maximum and/or mean height of the respiratory therapy device, in particular of the housing system, is greater than the maximum and/or mean length and/or maximum and/or mean width of the respiratory therapy device, in particular of the housing system. In the vertical mounting position, the maximum and/or mean height of the respiratory therapy device, in particular of the housing system, is greater than the maximum and/or mean length and/or maximum and/or mean width of the second mounting surface on which the respiratory therapy device is set in the vertical mounting position.

In particular, the mounting surfaces are arranged relative to each other at an angle of at least 65° and at most 115° and preferably at an angle of at least 80° and at most 100° and particularly preferably at an angle of 90°+/−5°.

It is also preferable that the mounting surfaces are arranged at right angles to each other. In particular, the first mounting surface is arranged at one of above-described angles to the second mounting surface. In such an embodiment, the advantages of the various mounting positions are seen particularly clearly. Other angles of the mounting surfaces relative to each other are also possible.

In particular, a device center of gravity of the respiratory therapy device is arranged in a lower half of the device and preferably in a lower quarter and particularly preferably in a lower eighth of the device. The device center of gravity is particularly preferably arranged at least in the one of the at least two mounting positions, as described above, in which the respiratory therapy device has a greater mounting height. In particular, the device center of gravity is arranged in this way at least in the vertical mounting position. In particular, the device center of gravity is arranged in this way in the mounting position which has a smaller mounting surface than the other mounting position. It is also possible that the device center of gravity is arranged in this way in all of the intended mounting positions. In particular, the device center of gravity, at least in the vertical mounting position, is arranged closer to the second mounting surface than to an upper end of the device. It is in this way possible to reliably counteract accidental overturning of the device.

The device center of gravity is provided in particular at least by the arrangement of at least one battery system for the energy supply of the respiratory therapy device and/or by the arrangement of at least one fan system for generating a respiratory gas stream. The device center of gravity can also be provided by the arrangement of at least one other heavy device component. Such embodiments favor a low center of gravity, particularly in the vertical arrangement. The battery system serves in particular to supply energy during ventilation and/or during a cough maneuver and in particular provides the energy for the respiratory therapy.

The respiratory therapy device comprises in particular at least one interface for coupling hoses for a respiratory gas stream and, for example, a breathing hose and/or cough hose. The respiratory therapy device comprises in particular at least one cable connection for an electrical energy supply and/or for mains connection or the like. The interface and/or the cable connection can also be of suitable design for connecting accessories for the respiratory therapy device. The interface and/or the cable connection are preferably arranged in a lower device half, at least in the one of the at least two mounting positions in which the respiratory therapy device has a greater mounting height. In particular, the interface and/or the cable connection are arranged in a lower quarter and particularly preferably in a lower eighth of the device. Such an arrangement of the interface and/or of the cable connection is provided in particular in the vertical mounting position. Such an arrangement of the interface and/or of the cable connection can also be provided in both mounting positions. In particular, the interface and/or the cable connection, at least in the vertical mounting position, are arranged closer to the second mounting surface than to an upper end of the device. It is thus possible to reliably avoid a situation in which accidental pulling on a hose or cable creates excessive torques, which could cause the device to overturn.

The interface and/or the cable connection are preferably arranged laterally on the housing system in all of the intended mounting positions. In particular, the interface and the cable connection, in all of the intended mounting positions, preferably at least in the vertical mounting position, are located laterally with respect to the mounting surfaces. It is thus possible to particularly effectively counteract overturning caused by accidental pulling.

The respiratory therapy device comprises in particular at least one handle system. The handle system is used in particular for carrying the device and/or for changing the mounting position. In particular, the handle system is arranged in such a way that the handle system is located, in a horizontal mounting position, on the front face, and, in a vertical mounting position, on a top face of the housing system. In this way, the handle system can be reached, in particular in both mounting positions, by an operator standing in front of the respiratory therapy device. In particular, the handle system can be reached in both mounting positions from the same user position. In particular, the handle system is located, in both mounting positions, at a distance from the plane on which the respiratory therapy device is placed. This allows the handle system to be grasped or taken hold of in all of the mounting positions. The handle system can be grasped and/or at least taken hold of in all of the intended mounting positions.

The handle system is preferably arranged in a recessed manner on the housing system. In this way, the handle system preferably does not protrude from a contour of the housing system. In particular, the handle system is integrated in the housing system and, for example, embedded therein. Thus, the handle does not get in the way in any of the mounting positions. The handle system can also be fastened to the housing system in a projecting manner.

The handle system is preferably arranged immovably on the housing system. In particular, the handle system is rigid. Such an embodiment has the advantage that the device can be moved, and the mounting position changed, without the handle first having to be brought to a defined position and, for example, folded out. Moreover, a handle designed in this way allows both the carrying of the device and the quick change of position from the horizontal to the vertical mounting position, and vice versa, to be effected in a simple and ergonomic manner. However, it is also possible for the handle to be foldable and/or extendable and/or tiltable and/or pivotable or movable.

The housing system comprises in particular at least one housing edge, which is designed as a tilting edge or at least comprises one such edge. The housing system is in particular tilted via the tilting edge in order to change between the mounting positions. The tilting edge is preferably resilient and/or reinforced and in particular abrasion-resistant and/or rounded. It is also possible that the tilting edge is designed to be exchangeable and/or hardened. It is possible that the tilting edge comprises at least one protective element. The protective element is designed in particular to be soft and/or elastic and/or flexible or also hardened and/or exchangeable. Such an embodiment has the advantage that the device can be tilted without having to be lifted completely. Moreover, such a tilting edge allows the tilting process or the change of position to be effected without, for example, causing scratches to the device or to a mounting surface or without the user having to worry about making such scratches. The tilting edge extends in particular between the two mounting surfaces. In particular, the two mounting surfaces run together or toward each other at the tilting edge.

Such embodiments can also be provided at least in some areas for the mounting surfaces, in order to avoid wear or damage there. It is possible that the mounting surfaces are at least in part resilient and/or reinforced and in particular resistant to abrasion and/or equipped with at least one protective element.

In particular, the handle system is arranged opposite the tilting edge. In this way, a correspondingly large lever is in particular achieved, such that the device can be tilted particularly easily in order to change the mounting position.

The mounting surfaces are in particular provided by in each case at least one side face of the housing system. In particular, the mounting surfaces are each provided by an entire side face. This offers particularly large mounting surfaces, such that particularly reliable tilting stability is achieved. It is also possible that the mounting surfaces are provided by at least 75% and in particular at least 85% and preferably at least 90% and particularly preferably at least 95% of the respective side face. The side face is in particular an outer face. A side face is understood to mean, in particular, a front face, top face, bottom face, rear face, right-side face and/or left-side face.

Particularly preferably, the respiratory therapy device is suitable and designed to be transferred as intended between the mounting positions simply by mounting and preferably by tilting the housing system and in particular without further measures. In particular, no adjustments are needed to the housing system and/or the handle system. In particular, no coupling and/or adjustment and/or modification of components on the respiratory therapy device is necessary in order to transfer the respiratory therapy device between the mounting positions. In particular, no hoses and/or cables or the like have to be reconnected. In particular, the respiratory therapy device can be transferred between the mounting positions without the entire respiratory therapy device having to being lifted up. In particular, the respiratory therapy device can be transferred between the mounting positions simply by tilting or pivoting, in particular via the tilting edge. This offers particularly convenient adaptation of the mounting position.

Provision can be made that during operation, for example during ventilation and/or cough assistance, the respiratory therapy device can be transferred as intended between the mounting positions without further measures. In particular, the therapy does not have to be interrupted for this purpose. It is also possible that a pause in therapy is set automatically or manually for the transfer between the mounting positions.

In a particularly advantageous embodiment, the respiratory therapy device is suitable and designed to independently detect the adopted mounting position by means of at least one control system. In particular, the respiratory therapy device is suitable and designed, preferably by means of the control system, to change and/or monitor at least one device setting in accordance with the mounting position. It is possible that the control system is assigned at least one sensor system for detecting the mounting position. Additionally or alternatively, by means of the control system, at least one user information notice can be output in accordance with the mounting position, for example a notice that a device setting requires manual adjustment. The device setting can involve device components of the respiratory therapy device and/or therapy settings for ventilation or for cough therapy.

The respiratory therapy device is particularly preferably suitable and designed, by means of the control system, to set at least one display mode of a display system and/or at least one function assignment of an operating system in accordance with the mounting position. By means of such automatic adaptation of the display or operation, the flexible mounting of the device is even more convenient. In particular, the alignment of the display system and/or of the operating system is adjusted, in accordance with the mounting position, such that the display and the operating elements are legible. In particular, the alignment of the display system and/or of the operating system is rotated and/or mirrored. The operating system and/or the display system can also be rotated manually via at least one user input.

In particular, the respiratory therapy device comprises at least one display system and/or at least one operating system. The display system is in particular designed as a display screen or comprises at least one such screen. The operating system can comprise at least one and preferably a plurality of hard keys and/or soft keys. In particular, the operating system is designed as a user interface or comprises at least one such interface. It is possible that the operating system is integrated into the display system. For example, a display system with a touch-sensitive surface, also referred to as a touchscreen, can be provided. In particular, the touchscreen extends over the entire display system. In particular, a capacitive operating system is provided. The display system can be designed as an operating and display system. By way of such a touch-sensitive surface, a large number of soft keys in particular are provided. In particular, the alignment and/or assignment of the soft keys is adjustable in accordance with the mounting position. Additionally or alternatively, at least one operating system designed separately from the display system can be provided, which preferably comprises hard keys and, for example, buttons or switches. The hard keys are in particular arranged on a side face of the housing system that is not already occupied by the display system and/or a mounting surface.

The display system and/or operating system is preferably arranged and in particular integrated on at least one side face of the housing system. This side face is preferably the largest side face of the housing system in terms of area. It is possible and also preferable that the display system and/or the operating system is arranged on at least one side face of the housing system that has a larger area than the individual faces of the mounting surfaces. In this way, the information on the display can be identified particularly easily and presented clearly.

The side face with the display system and/or operating system is in particular at least 1.5 times as large and preferably twice as large as the surface area of the second mounting surface. The side face with the display system and/or operating system is in particular at least 1.1 times and preferably at least 1.3 times larger than the surface area of the first mounting surface. The side face with the display system and/or operating system can be at least equally as large as the surface area of the first mounting surface. The display systems or operating systems can also be arranged on a smaller side face. Display systems and/or operating systems can also be provided on at least one further side face.

In a particularly advantageous embodiment, provision is made that the display system occupies at least 70% and in particular at least 95% of the side face of the housing system. It is possible and preferable that the display system occupies up to 100% of the side face. It is also possible that the display system extends beyond the side face, on which it is arranged, onto at least one adjacent side face. The details given here relate to the arrangement and size of the display system in particular on its visible surface or display surface.

In a particularly advantageous development, the operating system can be actuated at least in an upper quarter and preferably at least in an upper eighth with lower forces than the forces that are necessary for tilting the housing system. The operating device is in particular integrated into the display system and is designed, with the latter, as a touch-sensitive surface or touch screen. In particular, the operating system comprises soft keys for this purpose. The operating forces described above are preferably also provided in the case of an operating system with hard keys. It is possible that hard keys are also arranged outside the display system and can be actuated with the above-described operating forces. As has been described above, the center of gravity is preferably also such that the forces required to tilt the housing system are correspondingly large. Reliable operation of the device is thus also possible in the vertical position without any risk of tilting caused by operating forces.

In all of the embodiments, it is preferred that the display system is inclined relative to the first mounting surface by between 0° and 55° and particularly preferably by between 10° and 45°. It is also preferred, in all of the embodiments, that the display system is inclined, relative to the second mounting surface, by between 40° and 95° and particularly preferably by between 45° and 85°. In the vertical mounting position, the display system is inclined in particular by 10° to 45° relative to the vertical. In the horizontal position, the display system is inclined in particular by 45° to 85° relative to the vertical. Other angular positions of the display systems are also possible. It is also possible that the display system is adjustable in its angle. A viewing angle of the display system in the horizontal mounting position is preferably adapted or optimized for a person who is standing. Preferably, a viewing angle of the display system in the vertical mounting position is adapted for a person who is lying down or seated. As a result, the device simply has to be tilted or placed down when the user lies down in bed or sits down. In one embodiment, at least one further operating element is arranged in the respiratory therapy device, on at least one of the side faces that are smaller in area.

At least one or two further operating elements are preferably located on the side face of smaller surface area, in which the handle system can also be arranged. It is preferable in turn that an operating element is arranged on each of the two sides of the handle system, wherein particular functions can be controlled with these operating elements. These functions can be, for example, the switching on/off of the device, the starting/stopping of therapy and/or the acknowledgement or muting of alarms. By arranging these special functions—depending on the chosen position of the device—on a side face particularly facing the user or on a side face directed substantially upward, these functions are particularly directly and easily accessible to the user.

In all of the embodiments, the respiratory therapy device is suitable and designed to make available a respiratory air stream required for operation, independently of the mounting position. In particular, the respiratory air stream required for operation can be made available in all of the intended mounting positions. In particular, all of the intended forms of therapy can be carried out in all of the intended mounting positions. The respiratory air stream is made available in particular by the fan system. In particular, at least one suction region of the fan system is designed in such a way that a respiratory air intake functions in all of the intended mounting positions and, in particular, there is no risk of blockage.

It is preferred that all of the openings provided in the housing system are protected, in all of the intended mounting positions, at least against solid foreign bodies with a diameter of greater than or equal to 12.5 mm and/or against access with a finger and/or have protection against falling water when the housing system is inclined by up to 15°. In particular, the device or the housing system meets at least the protection class IP22, valid on the filing date, or a higher protection class.

The respiratory therapy device is particularly preferably designed as a portable respiratory therapy device. In particular, the respiratory therapy device has a maximum weight of 10 kg and particularly preferably a maximum weight of 6 kg. In particular, the respiratory therapy device can be carried by means of at least one handle system. In particular, the respiratory therapy device has a mains-independent energy supply by means of at least one battery system. In particular, the respiratory therapy device has a weight of between 1 kg and 10 kg and preferably of between 1.5 kg and 6 kg. In such an embodiment, the advantages of the various mounting positions become particularly clear. It is also possible that the respiratory therapy device is designed as a stationary device.

The housing system is preferably suitable and designed to be mounted and operated, in at least one of the mounting positions, with a housing system of a further respiratory therapy device according to the invention in a mutually supportive arrangement, also referred to as a back-to-back arrangement. Preferably, the mounting surfaces of the respective housing systems complement each other to form a common mounting surface. The first mounting surface is supported in particular on the first mounting surface of the other housing system. The housing systems preferably abut each other via the mounting surfaces provided for the horizontal mounting position. In particular, the respiratory therapy devices can jointly support each other. This affords a particularly stable and tilt-proof arrangement of the respiratory therapy devices. In particular, the housing systems are located in the same mounting position, for example both vertical or both horizontal.

A mutually supportive arrangement is particularly preferably provided in the vertical mounting position. The mutually supportive arrangement can also be provided in the horizontal mounting position. However, it is also possible that, in the mutually supportive arrangement, one device is in the vertical position and the other device is in the horizontal position. It is possible that the respiratory therapy devices are operatively connected when in a mutually supportive arrangement. For example, in such an arrangement, the control systems can be coupled to one another in a wireless and/or wired manner.

The applicant reserves the right to claim an assembly which is composed of at least two respiratory therapy devices of the kind described above. The two respiratory therapy devices can be mounted and preferably operated by means of their housing system in the mutually supportive arrangement. It is possible that the two respiratory therapy devices in this arrangement automatically establish an operative connection with each other. In particular, one respiratory therapy device is designed or can be operated as a cough assist device and the other respiratory therapy device as a ventilator.

Particularly preferably, the housing system is suitable and designed to be fastened to a wheelchair and/or to a hospital bed at least in the vertical mounting position. In particular, the housing system favors such an arrangement. Particularly preferably, the housing system is fastened in the vertical mounting position to the back of a wheelchair. For this purpose, the corresponding mounting surfaces can be equipped with at least one support system.

The respiratory therapy device is preferably designed as a cough assist device. The respiratory therapy device can preferably also be designed as a ventilator. In particular, the respiratory therapy device is suitable and designed to be used both as a cough assist device and as a ventilator. In such an embodiment, the invention can be used particularly advantageously.

The coupling system according to the invention serves for coupling at least two respiratory therapy devices, wherein the respiratory therapy devices are preferably designed as has been described above. The coupling system comprises at least two coupling units which can be connected to each other via a coupling element. A first respiratory therapy device can be received horizontally on at least one coupling unit by means of its housing system. A second respiratory therapy device can be received vertically on at least one other coupling unit by means of its housing system. In this way, the respiratory therapy devices in different mounting positions, for example a horizontal position and a vertical position, can simultaneously be received on the coupling system and in particular operated as intended thereon.

Such a coupling system permits particularly convenient and easily accessible and at the same time clear operation of the two respiratory therapy devices. Thus, the devices can be mounted in a particularly space-saving manner by virtue of the vertical and horizontal arrangements. Such a coupling system is particularly advantageous for example when a patient has to be managed with a cough assist device and at the same time with a ventilator. Moreover, the advantages of the respiratory therapy device according to the invention become particularly clear in combination with the coupling system according to the invention.

In particular, the mounting surfaces of the housing systems are suitable and designed to be coupled to the coupling system or to be fastened to the coupling system. In particular, both the horizontal coupling and the vertical coupling of the respiratory therapy device in question take place via the first mounting surface thereof. In particular, the first mounting surface is suitable and designed to be coupled both to the first and to the second coupling unit. In particular, the vertical respiratory therapy device is coupled to the coupling system via the first mounting surface, and in particular the horizontal respiratory therapy device is also coupled to the coupling system via its first mounting surface. It is possible that, additionally or alternatively, a coupling to the coupling system via the second mounting surface is provided. It is possible that the horizontal positioning takes place via the first and/or second mounting surface. It is also possible that the vertical positioning takes place via the first and/or second mounting surface.

The coupling units in particular comprise in each case at least one coupling sword and in each case at least one coupling plate. The coupling swords are in particular fastened to the coupling element. The coupling plates can in particular be fastened to the housing systems of the respiratory therapy devices. For fastening the respiratory therapy device to the coupling system, the coupling sword can preferably be inserted into the coupling plate. The connection of coupling sword and coupling plate can be released in particular without tools and can preferably be secured or locked by at least one securing system, for example by latching or the like. A reverse arrangement is also possible, in which the coupling swords are fastened to the housing systems of the respiratory therapy devices and the coupling plates are fastened to the coupling element. According to the invention, a coupling sword or a coupling plate is understood to mean any geometry that enables a suitable and releasable connection.

The coupling plate and the coupling sword can be made available by the coupling system or also by the respiratory therapy device. For example, the coupling plate can be permanently fitted on the respiratory therapy device or integrated in the housing system. However, it is also possible that the coupling plate is an accessory that can be retrofitted on the housing system. The coupling sword can be connected to the coupling element rigidly or also releasably.

It is possible that at least one coupling unit, preferably the one for coupling the horizontal respiratory therapy device, is equipped with at least one auxiliary handle. The auxiliary handle serves in particular for moving the coupling system with one or two respiratory therapy devices. For example, the coupling system can for this purpose be equipped with rollers or wheels in order to be able to conveniently move the whole assembly via the auxiliary handle.

The coupling system can be fastened in particular to a wheelchair and/or a hospital bed and/or another mobile or stationary system. The coupling system can also be suitable and designed to be placed on a support base, for example a table or a cabinet.

It is possible that, by means of at least one control system, the respiratory therapy devices are suitable and designed to automatically detect a coupling to the coupling system. In particular, at least one device setting can be changed by means of the control system in accordance with the detected coupling. For example, the operating system and/or display system can be aligned. It is also possible that, depending on the coupling, one of the respiratory therapy devices is chosen and operated as a cough assist device and the other respiratory therapy device is chosen and operated as a ventilator. It is possible that the respiratory therapy devices coupled via the coupling system at least partially complement each other in their operation. For example, the display system can be extended over both respiratory therapy devices. The respiratory therapy devices coupled via the coupling system can be operatively connected by means of the control system or can also be operated without operative connection.

The coupling element is in particular designed as at least one tube element or comprises at least one such tube element. In particular, the respiratory therapy devices can be fastened by means of the coupling units to different long sides of the coupling element and preferably of the tube element. The tube element is designed in particular as a polygonal tube and preferably as a square tube. The coupling element can be or comprise a profile element. The tube element has in particular a cavity which is designed as a receptacle, for example for respiratory therapy accessories or consumables.

It is possible that the coupling element is equipped with at least one longitudinal extension. The coupling unit of the horizontal respiratory therapy device is in particular arranged on the extension.

The coupling units are in particular arranged at an angle of 65° to 115° and preferably at an angle of 80° to 100° and particularly preferably at right angles to each other. In particular, the coupling units are arranged in this way when the two respiratory therapy devices are coupled as intended to the coupling system. In particular, the coupling units are arranged in this way on the coupling element and preferably on the tube element. In particular, the coupling swords and/or coupling plates are arranged in this way.

The applicant reserves the right to claim an assembly composed of two respiratory therapy devices according to the invention and of a coupling system according to the invention. Such an assembly also achieves the aforementioned object in a particularly advantageous manner and offers particularly convenient and user-friendly use of respiratory therapy devices. The assembly can be particularly advantageously used with a cough assist device and with a respiratory therapy device designed as a ventilator.

The mounting surfaces are in particular flat or planar. The mounting surfaces can also have elevations and/or depressions in some parts. For example, the mounting surfaces can be equipped with feet and/or bases or the like. The mounting surfaces are in particular arranged on the outside of the housing system. According to the invention, the mounting surface is understood to mean the surface that is directed down toward the mounting location. In particular, the respiratory therapy device is mounted in place by means of the mounting surfaces being placed on a support base and, for example, a table or a floor or a storage surface or the like. The mounting surfaces are in particular arranged adjacent to the housing system. The mounting surfaces can be made available by adjacent side faces, or also by side faces that are spaced apart by at least one further side face. The mounting surface is in particular integrated in the housing system and/or connected in one piece to the housing system. It is also possible that the mounting surface is designed separately from the housing system and, for example, is releasably or permanently fastened thereto.

The respiratory therapy device comprises in particular at least one electrically operable or operated respiratory gas source. In particular, the respiratory gas source comprises at least one fan system and/or blower system or is designed as such. The respiratory gas source is in particular accommodated by the housing system. The respiratory therapy device can in particular be operated electrically. A mains-connected and/or battery-operated operation can be provided.

Further advantages and features of the present invention will become clear from the description of the exemplary embodiments, which are explained below with reference to the accompanying figures.

In the figures:

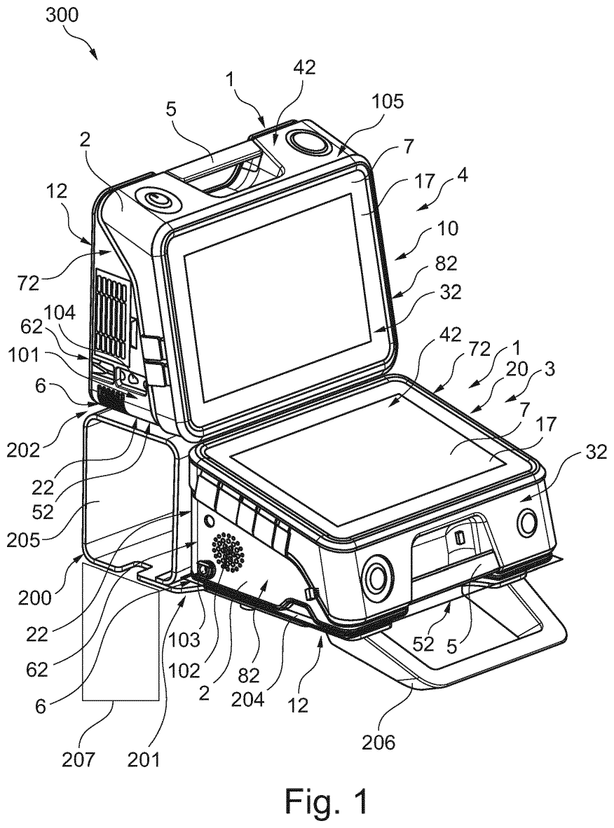

FIG. 1 shows a purely schematic representation of an assembly having a coupling system according to the invention with two respiratory therapy devices according to the invention in a perspective view;

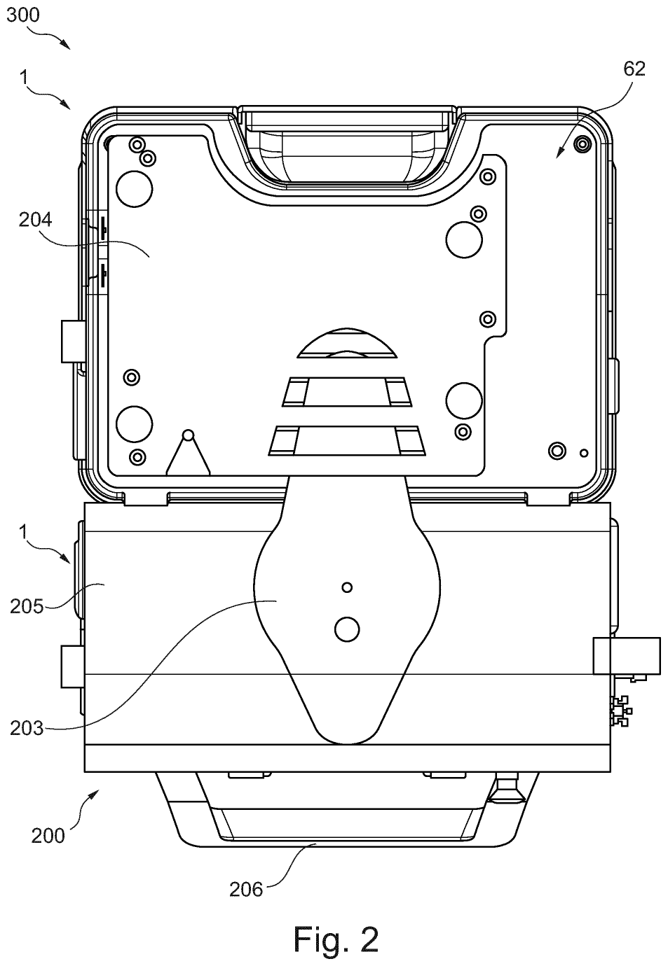

FIG. 2 shows the assembly of FIG. 1 in a view from behind;

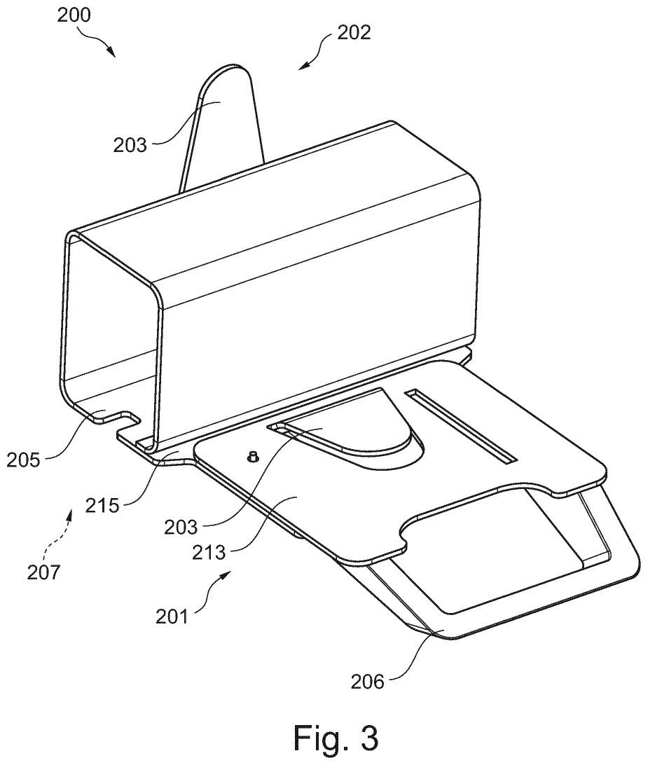

FIG. 3 shows a respiratory therapy device according to the invention in a perspective view;

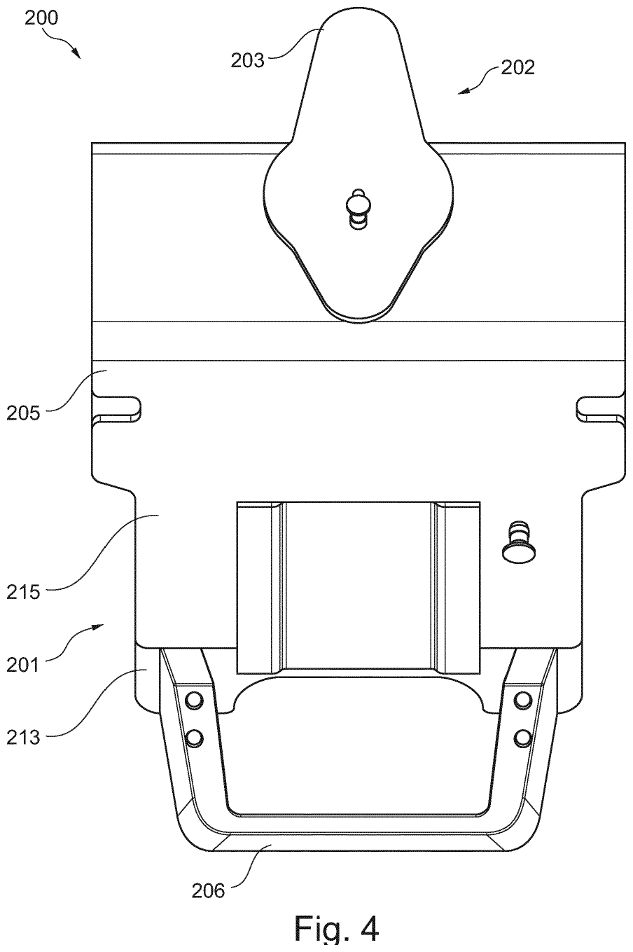

FIG. 4 shows a coupling system according to the invention in a perspective view;

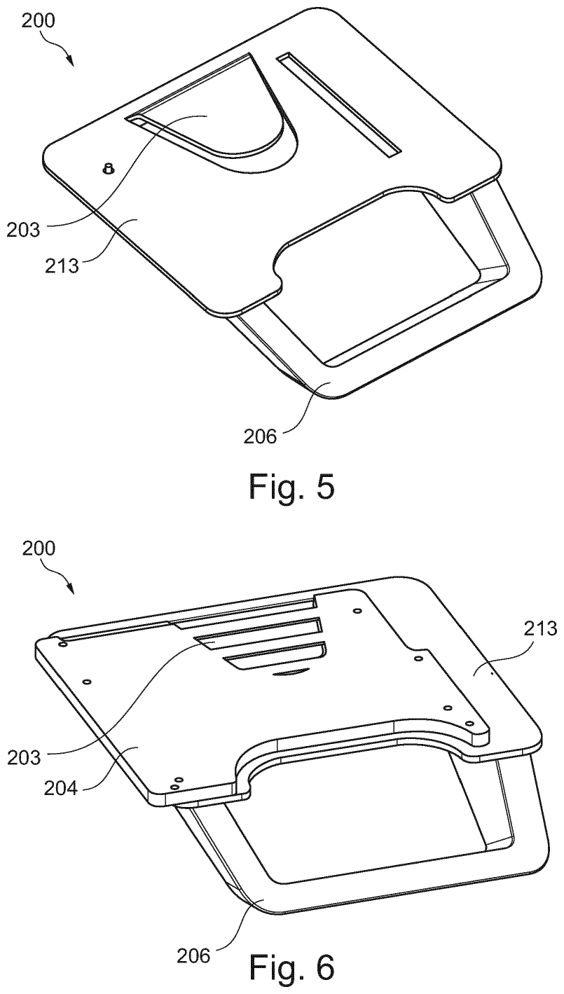

FIG. 5 shows the coupling system of FIG. 4 in a view obliquely from below;

FIG. 6 shows a perspective detailed representation of the coupling system of FIG. 4, in a view obliquely from above;

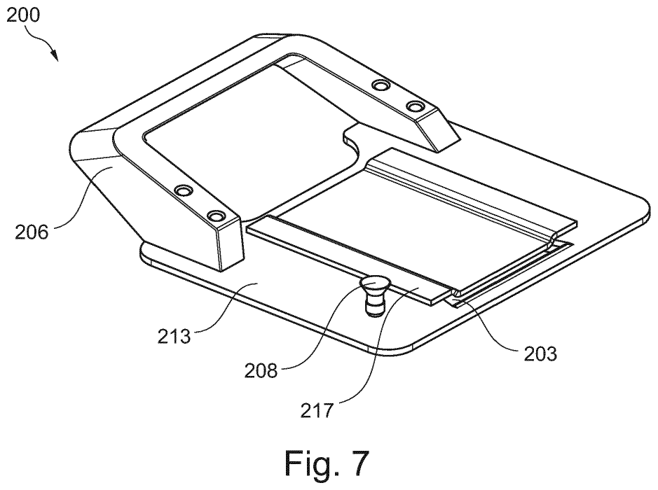

FIG. 7 shows another perspective and detailed representation of the coupling system of FIG. 4, in a view obliquely from above; and

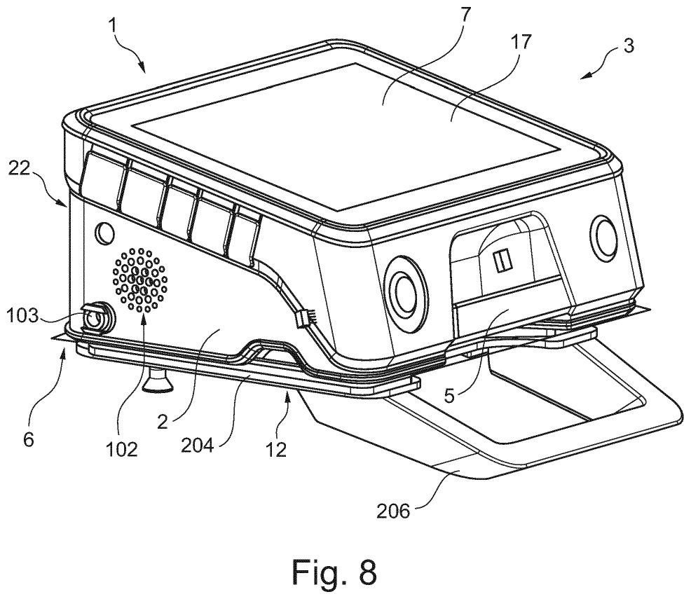

FIG. 8 shows a perspective and detailed representation of the coupling system of FIG. 4, in a view obliquely from below.

FIG. 1 shows two respiratory therapy devices 1 according to the invention, which are coupled here via a coupling system 200 according to the invention to form an assembly 300. Such an assembly 300 provides a particularly user-friendly and space-saving arrangement of two respiratory therapy devices 1. The respiratory therapy devices 1 can also be used individually or on their own (stand alone) without the coupling system 200. A single respiratory therapy device 1 according to the invention is shown in FIG. 8.

One respiratory therapy device 1 is designed here as a ventilator 20, and the other as a cough assist device 10. The ventilator 20 here comprises a fan system 102 (not visible here since arranged in the interior) in order to generate a respiratory air stream for ventilating the patient. The cough assist device 10 serves to specifically support the discharge of secretions from the airways of a patient and uses the fan system 102 for insufflation and exsufflation.

The internal device components of the respiratory therapy device 1 are accommodated here by a housing system 2. The housing system 2 here comprises a plurality of side faces, namely a front face 32, a top face 42, a bottom face 52, a rear face 62, and two laterally arranged sides 72, 82. The device 1 is equipped here as a portable respiratory therapy device with a maximum weight of 6 kg.

In addition, the respiratory therapy device 1 here comprises a display system 7 and an operating system 17. Part of the operating system 17 is made available by a touch-sensitive surface of the display system 7. The operating system 17 additionally comprises various hard keys, for example buttons and switches.

The respiratory therapy device 1 can be set up and operated in two different mounting positions 3, 4. The lower respiratory therapy device 1 of the assembly 300 is situated here in a horizontal mounting position 3, and the upper respiratory therapy device 1 in a vertical mounting position 4. To permit these mounting positions 3, 4, the housing system 2 here comprises two mounting surfaces 12, 22. A first mounting surface 12 is provided for the horizontal mounting position 3, and a second mounting surface 22 is provided for the vertical mounting position 4.

In the horizontal device 1, the first mounting surface corresponds to the bottom face 52, and the second mounting surface 22 corresponds to the rear face 62. In the vertical device 1, the first mounting surface 12 corresponds to the rear face 62, and the second mounting surface 22 corresponds to the bottom face 52.

In order to bring the individual respiratory therapy device 1 without the coupling system 200 from the horizontal to the vertical mounting position 3, 4 or vice versa, it merely has to be tilted. The change between the mounting positions 3, 4 is possible without any further measures, such as elements that can be coupled on or folded out. This is achieved here by, among other things, the fact that the mounting surfaces 12, 22 are designed to be sufficiently large and that a device center of gravity is also sufficiently low for the vertical position. Moreover, for the change of position, there is no need for hoses or cables to be reconnected. Furthermore, the respiratory therapy device 1 can provide the respiratory air stream required for the respective therapy independently of the mounting position 3, 4. Moreover, the openings provided for suctioning in of the air are arranged such that, in all mounting positions 3, 4, respiratory air suction is reliably permitted and there is no risk of blockage. The opening necessary for the suction and all the other openings of the housing system 2 correspond here to protection class IP22.

The device center of gravity in the vertical mounting position 4 is arranged here in a lower half of the device. For this purpose, a battery system 101 for energy supply and also the fan system 102 were arranged in the lower half of the device. Moreover, the interface 103 for the coupling of ventilation hoses and the cable connections 104 were arranged in the lower half of the device. Thus, the device 1 remains stable in the corresponding mounting position 3, 4 even if a hose or cable is accidentally pulled. The interfaces 103 and the cable connections 104 were moreover arranged in such a way that they are located laterally on the housing system 2 in all of the mounting positions 3, 4.

To make it possible to change between the mounting positions 3, 4 in a way that is as straightforward and convenient as possible, an integrated handle system 5 is provided here, which is arranged in a recessed manner on the housing system 2. As a result, the handle system 5 does not protrude from the contour of the housing system 2. Moreover, the handle is designed and arranged such that it can be grasped in both mounting positions 3, 4. Moreover, the handle system 5 is designed to be rigid and therefore does not have to be folded over or folded out before the mounting position 3, 4 is changed. The handle system 5 thus allows the device 1 to be carried in a simple and ergonomic manner and also enables the quick change of position from the horizontal to the vertical position 3, 4, and vice versa. Moreover, the handle system 5 can be reached in both mounting positions 3, 4 by a user standing in front of the respiratory therapy device 1.

To further simplify the mounting or laying down of the device 1, the housing system 2 is here equipped with a housing edge designed as a tilting edge 6. The device 1 can thus be tilted via the lower edge or rear edge for the change of position, without the user having to lift the whole device 1 up for this purpose. The tilting edge 6 is preferably provided, on the two mounting surfaces 12, 22, with suitable protective, soft or flexible mounting elements which allow the tilting process and the change of position to be carried out without, for example, causing scratches on the device or on a mounting surface 12, 22 or without risk of the user making such scratches.

The respiratory therapy device 1 here comprises a control system 105 which independently detects the respective mounting position 3, 4, for example by means of a sensor system, and then for example changes a device setting. For example, depending on the mounting position 3, 4, the alignment of the touchscreen is rotated or mirrored accordingly.

The display system 7 here has a display area which substantially covers the entire surface area of the largest side face of the device 1. This corresponds here, in the horizontal position 3, to the top face 42 and, in the vertical position 4, to the front face 32. The viewing angle of the display system 7 is optimized here in the horizontal mounting position 3 for a person who is standing. In the vertical mounting position 4, the viewing angle is then accordingly optimized for a person who is lying down or seated.

The respiratory therapy devices 1 shown here can also be mounted and operated in a back-to-back arrangement without the coupling system 200. For this purpose, both devices 1 are mounted in the vertical position 4 such that they stand on the second mounting surfaces 22. The first mounting surfaces 12, that is to say the rear faces 62, then touch each other, such that the devices can support each other at all times or in the event of an impact.

The coupling system 200 permits a mechanical and reliable connection of the two respiratory therapy devices 1. For this purpose, the horizontal respiratory therapy device 1 is joined to a coupling element via a first coupling unit 201, and the vertical respiratory therapy device 1 is connected to a coupling element via a second coupling unit 202. The coupling element 205 is designed here as a tube element and, for example, as a square tube.

In one embodiment, a chassis 207, shown here in a highly schematic manner, can be mounted on the coupling element 205. In this way, the entire assembly 300 can be moved in a particularly simple way and handled flexibly. To take hold of the assembly 300, an auxiliary handle 206 is then provided for example which, in the embodiment shown here, is arranged in the region of the horizontal respiratory therapy device 1.

The coupling system 200 or the assembly 300 are described below with reference to FIG. 1 and to the detailed representations in FIGS. 2 to 7.

The coupling units 201, 202 each comprise a coupling sword 203 and a coupling plate 204. The coupling swords 203 are attached here to the coupling element 205. The connection of coupling sword 203 and coupling plate 204 can be seen particularly well in FIG. 2, which shows this connection for the upper device 1 in a rear view of the assembly 300. The coupling plates 204 are here fastened as accessory parts to the first mounting surfaces 12 of the housing systems 2 of the respective respiratory therapy devices 1. By inserting the coupling sword 203 into the coupling plate 204, the devices 1 are fastened to the coupling element 205. Releasing and fastening without tools is preferably provided. Moreover, the connection can be secured, for example by means of a latch. Here, for example, a securing element 208 is provided, which can be seen particularly well in FIG. 7.

The coupling sword 203 for the lower or horizontal device 1 is fastened here to an extension 215 extending from a longitudinal side of the coupling element. The extension 215 can be seen particularly clearly in FIGS. 3 and 4, in which the coupling system 200 is shown without respiratory therapy devices 1.

As can be seen particularly clearly in FIG. 2, the coupling sword 203 for the upper device 1 is mounted on the coupling element 205. As can be seen particularly clearly in FIG. 3, the coupling sword for the lower device 1 is arranged on a support plate 213. The coupling sword 203 is then mounted on the extension 215 via the support plate 213.

In FIGS. 5 to 7, the support plate 213 with the coupling sword 203 and with the auxiliary handle 206 of the coupling system 200 is shown from different views. The support plate 213 of FIG. 6 is equipped in FIG. 7 with a coupling plate 204, such that the connection of coupling sword 203 and coupling plate 204 can be seen particularly clearly. The coupling plate 204 can be part of the coupling system 200 or can also be an accessory part or a fixed part of the housing system 2 of the respiratory therapy device 1. For example, the coupling plate 204 can be screwed onto the housing system 2. Alternatively, the coupling plate 204 can also be designed as a corresponding recess in the housing system 2, into which recess the coupling sword 203 can be inserted. Corresponding designs are also provided for the coupling plate 204 of the upper respiratory therapy device 1 as shown in FIG. 2.

The bottom face of the mounting plate 213 is shown in FIG. 7, such that a connecting system 217 can be seen particularly clearly here. The connecting system 217 can be provided to connect coupling plate 204 and coupling sword 203 or can also serve for connection to a chassis 207.

FIG. 8 shows a single respiratory therapy device 1 in a horizontal mounting position 3. The device 1 is also equipped here with a coupling plate and an auxiliary handle 206, which however belong to the above-described coupling system 200 and are not part of the respiratory therapy device 1 shown here. The device 1 shown here can be easily and conveniently transferred to the vertical mounting position 4 by being tilted about the tilting edge 6.

In one embodiment, the coupling system 200 can have a support plate 213 for a respiratory therapy device 1, wherein the coupling system 200 is mounted with the support plate 213 on a chassis 207. The support plate 213 has at least one connecting system 217, which can be designed as a (paired) rail system, wherein the connecting system 217 is set up and designed to interact, substantially by force-fit or form-fit engagement, with a corresponding receptacle on the respiratory therapy device 1. For this purpose, the respiratory therapy device has such receptacles, that interact with the rail system by force-fit or form-fit engagement, in the region of its mounting surface. The respiratory therapy device can be placed with its mounting surface onto the support plate and can be pushed on the support plate into a mounting position, wherein the respiratory therapy device moves at least in part along the rails until the rails interact (by force-fit or form-fit engagement) with the receptacles. A securing element 208 in the region of the support plate serves to secure the respiratory therapy device 1 on the support plate in the mounting position. The support plate is preferably arranged horizontally on a chassis, wherein the chassis can extend in the vertical direction relative to the support plate. The support plate has the rails as left and right boundaries, in the form of an elevation, and has, as its rear boundary, a further rail or edge or a section of the chassis. The front region of the support plate has no boundary and serves to place the respiratory therapy device 1 here and push it onto the support plate.

The connecting system 217 and the corresponding receptacle on the respiratory therapy device 1 are preferably set up and designed such that both align with each other or slide along each other when the respiratory therapy device is placed with its mounting surface onto the support plate and is pushed on the support plate into a mounting position. For this purpose, the rails are preferably arranged in a funnel shape on the horizontally arranged support plate, wherein the opening of the funnel is to the front and wherein the funnel narrows to the rear. The respiratory therapy device can then be locked on the support plate by means of a securing element 208.

### LIST OF REFERENCE SIGNS

1 respiratory therapy device
2 housing system
3 mounting position, horizontal
4 mounting position, vertical
5 handle system
6 tilting edge
7 display system
10 cough assist device
12 mounting surface
17 operating system
20 ventilator
22 mounting surface
32 front face
42 top face
52 bottom face
62 rear face
72 side
82 side
101 battery system
102 fan system
103 interface
104 cable connection
105 control system
200 coupling system
201 coupling unit
202 coupling unit
203 coupling sword
204 coupling plate
205 coupling element
206 auxiliary handle
207 chassis
208 securing element
213 support plate
215 extension
217 connecting system
300 assembly

1.-38. (canceled)

39. A respiratory therapy device for generating a respiratory air stream for carrying out respiratory therapy, wherein the device comprises at least one housing system for accommodating device components, the housing system comprising at least two mounting surfaces, such that the respiratory therapy device can be mounted and operated as intended in at least two mounting positions.

40. The respiratory therapy device of claim 39, wherein the housing system comprises at least one first mounting surface for at least one first mounting position and at least one second mounting surface for at least one second mounting position.

41. The respiratory therapy device of claim 40, wherein the first mounting surface is suitable and configured for a horizontal mounting position and the second mounting surface is suitable and configured for a vertical mounting position.

42. The respiratory therapy device of claim 41, wherein, in the horizontal mounting position, a mounting height of the respiratory therapy device is less than a length and/or a width of the first mounting surface, and/or wherein, in the vertical mounting position, a mounting height of the respiratory therapy device is greater than a length and/or a width of the second mounting surface.

43. The respiratory therapy device of claim 39, wherein the mounting surfaces are arranged at an angle of at least 65° and at most 115°.

44. The respiratory therapy device of claim 39, wherein a device center of gravity is arranged in a lower device half, at least in the one of the at least two mounting positions in which the respiratory therapy device has a greater mounting height.

45. The respiratory therapy device of claim 44, wherein the device center of gravity is made available at least by an arrangement of at least one battery system for energy supply to the respiratory therapy device and/or at least one fan system for generating a respiratory gas stream.

46. The respiratory therapy device of claim 39, wherein the device further comprises at least one interface for coupling a hose for a respiratory gas stream and/or at least one cable connection, the at least one interface and/or the at least one cable connection being arranged in a lower device half, at least in the one of the at least two mounting positions in which the respiratory therapy device has a greater mounting height.

47. The respiratory therapy device of claim 46, wherein the at least one interface and/or the at least one cable connection are arranged laterally on the housing system in all of the intended mounting positions.

48. The respiratory therapy device of claim 39, wherein at least one handle system is arranged in such a way that the handle system is located, in a horizontal mounting position, on a front face of the housing system, and, in a vertical mounting position, on a top face of the housing system, such that the handle system can be reached in both mounting positions by an operator standing in front of the respiratory therapy device.

49. The respiratory therapy device of claim 39, wherein at least one handle system is arranged in a recessed manner on the housing system, such that the handle system does not protrude from a contour of the housing system and/or at least one handle system is arranged immovably on the housing system.

50. The respiratory therapy device of claim 39, wherein the housing system comprises at least one housing edge designed as a tilting edge, the tilting edge being resilient and/or reinforced and/or rounded.

51. The respiratory therapy device of claim 39, wherein the mounting surfaces are made available by in each case a side face of the housing system.

52. The respiratory therapy device of claim 39, wherein the device is suitable and configured to be capable of being transferred as intended between the mounting positions by mounting of the housing system.

53. The respiratory therapy device of claim 39, wherein the device is suitable and configured to independently detect an adopted mounting position by at least one control system to change at least one device setting in accordance with the mounting position.

54. The respiratory therapy device of claim 53, wherein at least one display mode of a display system and/or at least one function assignment of an operating system is set in accordance with the mounting position.

55. The respiratory therapy device of claim 39, wherein all of the openings provided in the housing system are protected, in all of the intended mounting positions, at least against solid foreign bodies with a diameter of ≥12.5 mm and against access with a finger and have protection against falling water when the housing system is inclined by up to 15°.

56. A coupling system for coupling at least two respiratory therapy devices according to claim 39, wherein the coupling system comprises at least two coupling units which can be connected to each other via a coupling element, and wherein a first respiratory therapy device can be received horizontally on at least one coupling unit by means of its housing system and a second respiratory therapy device can be received vertically on at least one other coupling unit by means of its housing system, such that the respiratory therapy devices in different mounting positions can be received simultaneously on the coupling system and operated as intended thereon.

57. A coupling system for receiving a respiratory therapy device, wherein the coupling system comprises a support plate for a respiratory therapy device and is mounted with the support plate on a frame, the support plate comprising has at least one connecting system, the connecting system being set up and designed to interact, substantially by force-fit or form-fit engagement, with a corresponding receptacle on the respiratory therapy device, and wherein the respiratory therapy device for this purpose has, in a region of its mounting surface, such receptacles that interact by force-fit or form-fit engagement with the rail system, the respiratory therapy device being placed with its mounting surface on the support plate and being pushed into a mounting position on the support plate, wherein the respiratory therapy device moves at least in part along rails of the rail system until the rails interact with the receptacles (by force-fit or form-fit engagement), wherein the support plate is arranged horizontally on a frame, wherein the support plate has, as a left and right boundary, rails as connecting system, and wherein a front region of the support plate has no boundary, or only connecting device has a securing element for locking the respiratory therapy device.

58. An appliance, wherein the appliance comprises at least one coupling system for at least two respiratory therapy devices, at least one respiratory therapy device being a ventilator and at least one other respiratory therapy device being a cough assist device.
